(19) United States
(12) Patent Application Publication
(10) Pub. No.: US 2020/0123190 A1
(43) Pub. Date: Apr. 23, 2020

(54) 5'-POSITION DIBENZYL MONOPHOSPHATE DERIVATIVE OF NUCLEOSIDE-BASED ANTICANCER AGENT OR ANTIVIRUS AGENT

(71) Applicant: Ohara Pharmaceutical Co., Ltd., Koka-shi Shiga (JP)

(72) Inventor: Magoichi Sako, Koka-shi Shiga (JP)

(21) Appl. No.: 16/606,519

(22) PCT Filed: Apr. 24, 2018
(86) PCT No.: PCT/JP2018/016512

§ 371 Date: Oct. 18, 2019

(30) Foreign Application Priority Data
Apr. 25, 2017 (JP) 2017-085746

(51) Int. Cl. C07H 19/10; C07H 19/20; A61P 31/12; A61P 35/00

(52) U.S. Cl. CPC C07H 19/10; A61P 35/00; A61P 31/12; C07H 19/20

(57) ABSTRACT

To provide, in place of injected agents (nucleoside-based anticancer agents or antivirus agents) clinically used as therapeutic drugs for cancer or virus infections, a medicine that has high stability with respect to various hydrolytic metabolic enzymes, is absorbed into the body even by oral administration, and exhibits a cytocidal effect by being incorporated into a DNA and RNA biosynthetic route and inhibiting the modification and extension of DNA and RNA or inhibiting reverse transcriptases or inhibiting protein synthesis.

The aforementioned problem is solved by a novel compound represented by formula (I). (In the formula, D is the 5′-position moiety of a nucleoside-based anticancer agent or an antivirus agent, and R¹ and R² are each a benzyl group that may have the same substituent or different substituents.)

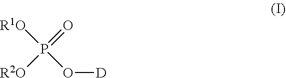

### TECHNICAL FIELD

The present invention relates to the development of a compound which has remarkable stability with respect to various hydrolytic metabolic enzymes and can be used as prodrug for 5′-position monophosphate of nucleoside-based anticancer agent or antiviral agent.

### TECHNICAL BACKGROUND

Examples of anticancer nucleosides currently used in clinical practice include Cytarabine (also called as Cytosine arabinoside and Ara-C, or by the product names of Cytosar-U® and Depocyt®), Floxuridine (by the product name of FUDR®), Pentostatin (also called as Deoxycoformycin, or by the product name of Nipent®), Fludarabine (by the product name of Fludare®), Cladribine (by the product name of Leustatie®), Gemcitabine (by the product name of Gemzar®), 5-Azacytidine (also called as Azacytidine, or by the product name of Vidaze®), 2′-Deoxy-5-azacytidine (also called as Decitabine, or by the product name of Dacogen®), Clofarabine (by the product names of Clolar® and Evoltra®), Nelarabine (by the product names of Arranon® and Atriance®), Trifluorothymidine (also called as TFT and Trifluridine, or by the product names of Viroptic® and)Lonsurf®, and the like. In frequently dividing cells, the 5′-position hydroxyl group of these nucleosides is monophosphateified by corresponding nucleoside kinase (2′-deoxycytidine kinase, thymidine kinase 1 & 2, or 2′-deoxyguanosine kinase) before being incorporated into DNA and RNA via nucleic acid biosynthetic route, and exhibits a cytocidal effect by inhibiting the modification and extension of DNA and RNA, and inhibiting the synthesis of corresponding protein. Therefore, these nucleosides are used as therapeutic agents for various cancers (non patent document 1).

Besides, examples of antiviral nucleosides which are currently used in clinical practice include Zidovudine (also called as ZDV, Azidothymidine and AZT, or by the product name of Retrovir®), Lamivudine (also called as 3TC, or by the product name of Epivir®), Stavudine (also called as Sanilvudine and d4T, or by the product name of Zerie®), Abacavir (also called as ABC, or by the product name of Ziagen®), Emtricitabine (also called as FTC, or by the product names of Emtriva® and Coviracil®), Didanosine (also called as ddI, or by the product name of Videx®), Zalcitabine (also called as ddC, or by the product name of Hivie®), and the like. In virus-infected cells, the 5′-position hydroxyl group of these nucleosides is monophosphateified by corresponding nucleoside kinase before being incorporated into DNA and RNA via nucleic acid biosynthetic route, and exhibits a cytocidal effect by inhibiting reverse transcriptase related to DNA and RNA synthesis. Therefore, these nucleosides are used as antiviral agents (non patent documents 2-3).

However, monophosphateification of the 5′-position hydroxyl group of these nucleosides is the most rate-limiting step during nucleic acid biosynthetic route. In addition, in case of long term use of these anticancer agents and antiviral agents, down-regulation of nucleoside kinases involved in the monophosphateification process is induced, which tends to cause drug resistance to these nucleoside-based anticancer agents and antiviral agents (non patent document 4).

Therefore, as medicines for clinical use, it is more desirable to use 5′-position monophosphates of these nucleoside-based anticancer agents and antiviral agents, that is, compounds at their mononucleotide level. However, all of these corresponding 5′-position monophosphates contain free phosphate residues which are so polar that they cannot easily pass through cell membranes in vivo. Therefore, it is not speculated that intended clinical effect can be expected by either administration method.

In this circumstances, various corresponding 5′-position monophosphates have been studied as various prodrugs for nucleoside-based anticancer agents and antiviral agents, such as use of functional groups containing an ester that are hydrolytically metabolized easily by carboxylesterase in the side chain as protecting groups for phosphate residue, and use of the functional group of phosphoamidite that is hydrolytically metabolized easily by phosphoamidase (non patent documents 5).

However, neither of them showed desirable clinical effect in many of these attempts because of extremely low stability with respect to various hydrolytic enzymes in blood or liver, and high cytotoxicity of compounds produced during deprotection. However, as the result of these attempts, some antiviral agents have become medicines that can be clinically used, for example, Tenofovir DF (by the product name of Viread®)(patent document 1), Pradefovir (also called as PDV, or by the product names of Remofovir® and Hepavir®) (patent document 2) and Sofosbuvir (by the product name of Sovaldi®) (patent document 3), and the like.

Therefore, as a prodrug for 5′-position monophosphate of nucleoside-based anticancer agent or antiviral agent, a derivative that has high stability with respect to various hydrolytic metabolic enzymes, that can be deprotected non-enzymatically or enzymatically in cells to easily release 5′-position monophosphate of nucleoside-based anticancer agent or antiviral agent, and that has low cytotoxicity of compounds produced during the deprotection is desirable.

### PRIOR ART DOCUMENTS

### Patent Documents

1. U.S. Pat. No. 5,977,089
2. Publication of WO patent No. 03095665
3. Publication of US patent No. 2010016251

### Non Patent Documents

1. Chemical Reviews, 2016, vol. 116, No. 23, p. 14379-14455
2. Clinical Microbiology Reviews, 2016, vol. 29, No. 3, p. 695-747
3. Medical Research Reviews, 2016, vol. 36, No. 6, p. 1127-1173
4. Biochemical and Biophysical Research Communications, 2012, vol. 421, No. 1, p. 98-104
5. Chemical Reviews, 2014, vol. 114, No. 18, p. 9154-9218

### SUMMARY OF THE INVENTION

### Problems to be solved by the Invention

An object of the present invention is to provide a derivative of 5′-position monophosphate of nucleoside-based anticancer agent or antiviral agent, that has high stability with respect to various hydrolytic metabolic enzymes, can be deprotected non-enzymatically or enzymatically smoothly in cells to be incorporated into nucleic acid biosynthetic route, and has low cytotoxicity of compounds produced during the deprotection.

### Solutions to the Problems

In order to provide more useful medicines as preventive or therapeutic agents for cancer or viral infection, the present inventors have earnestly undertaken studies on finding novel compounds that possess both excellent pharmacologic effects of having remarkable stability with respect to hydrolytic metabolic enzymes, such as cytidine deaminase, a metabolic enzyme, and being incorporated into nucleic acid biosynthetic route in vivo and excellent physicochemical properties. The present inventors have therefore synthesized various 5′-position monophosphate dialkyl derivatives of nucleoside-based anticancer agent or antiviral agent including 5-azacytidines. As the results of investigations on their chemical reactivity, it was found out unexpectedly that a 5′-position dialkyl monophosphate derivative of nucleoside-based anticancer agent or antiviral agent with specific structure had remarkable stability with respect to various hydrolytic metabolic enzymes, it was deprotected non-enzymatically or enzymatically smoothly in cells to be incorporated into nucleic acid biosynthetic route, and it had low cytotoxicity of compounds produced during the deprotection, which are excellent properties as a medicine. The present inventors continued the investigation and finally completed the present invention.

That is, the present invention has solved the above problems by providing the following invention.
[1] A compound represented by formula (I), or salt thereof,

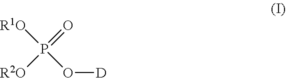

wherein D is 5′-position moiety of nucleoside-based anticancer agent or antiviral agent, and R¹ and R² are each a benzyl group that may have the same substituent or different substituents.
[2] The compound according to that described in [1], or salt thereof, wherein each of the R¹ and R² is a benzyl group which may have an alkyl or a halogen atom as a substituent.
[3] The compound according to that described in [2], or salt thereof, wherein the alkyl is C₁ to C₆ alkyl group.
[4] The compound according to that described in [2], or salt thereof, wherein the alkyl is methyl group or ethyl group.
[5] The compound according to that described in [2], or salt thereof, wherein the halogen atom is a fluorine atom, a chlorine atom or a bromine atom.
[6] The compound according to that described in [1], or salt thereof, wherein the R¹ and R² are benzyl groups.
[7] The compound according to that described in [1], or salt thereof, wherein the nucleoside-based anticancer agent shown as D is Cytarabine, Floxuridine, Pentostatin, Fludarabine, Cladribine, Gemcitabine, Clofarabine, Nelarabine, Trifluorothymidine, DFP-10917, Cordycepin, 8-Chloroadenosine, RX-3117, Triciribine, Forodesin, 5-Fluorodeoxycytidine, Ribavirin or Acadesine.
[8] The compound according to that described in [1], or salt thereof, wherein the antiviral agent shown as D is Zidovudine, Lamivudine, Stavudine, Abacavir, Emtricitabine, Didanosine or Stavudine.
[9] The compound according to that described in [1], or salt thereof, wherein the compound is

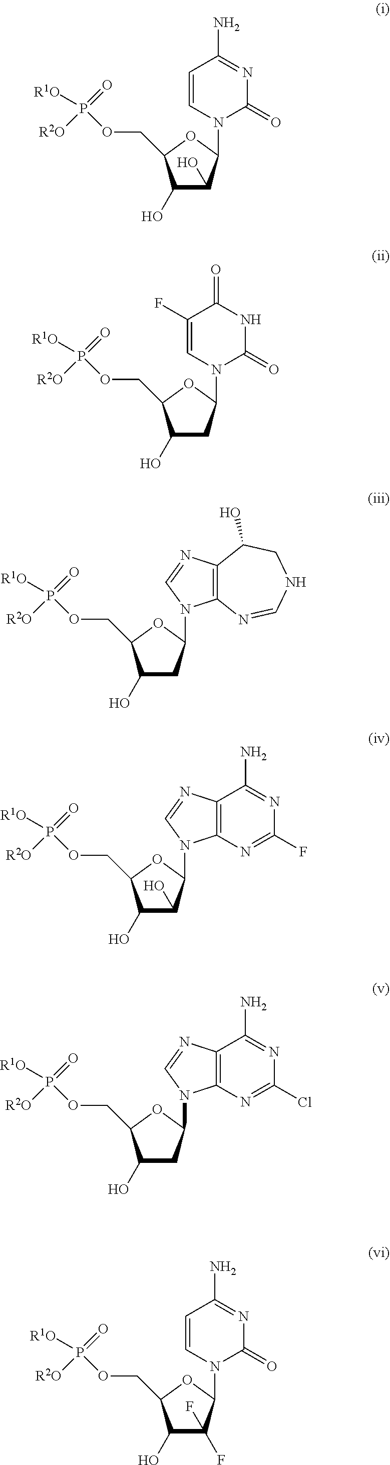

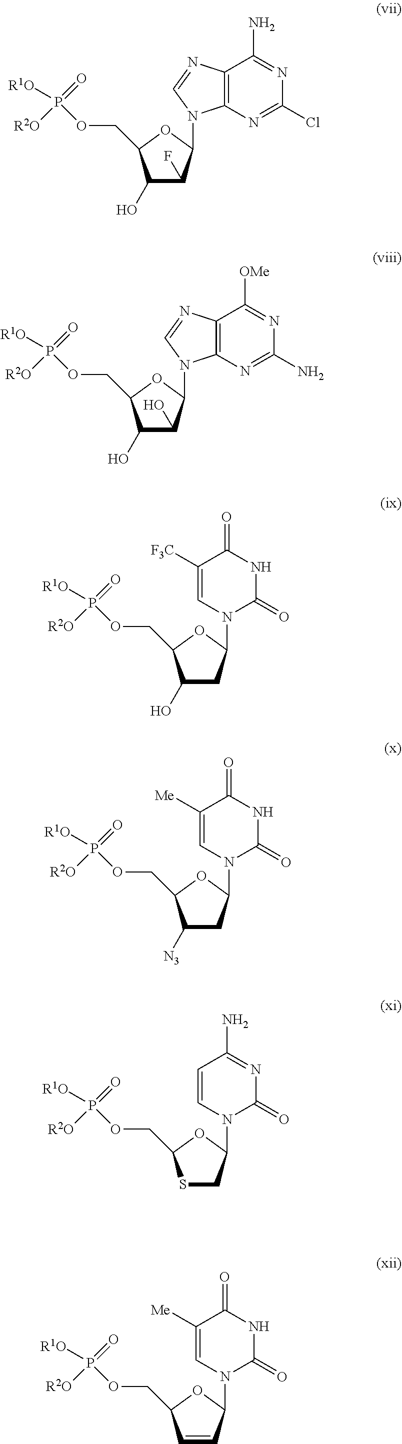

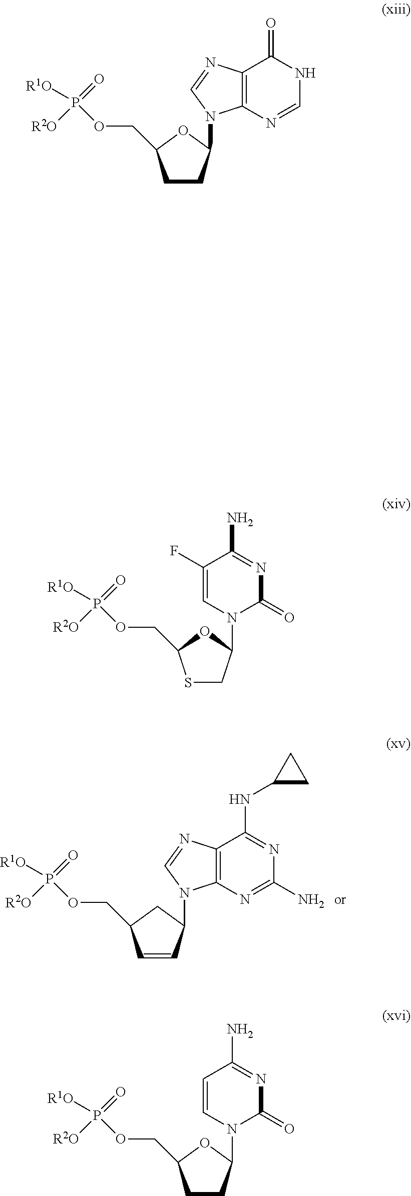

[10] A method for producing the compound according to that described in [1], or salt thereof, which comprises reacting a nucleoside-based anticancer agent or antiviral agent with phosphorus oxychloride and then reacting with an optionally substituted benzyl alcohol in the presence of a dehydrohalogenating agent, or comprises reacting a nucleoside-based anticancer agent or antiviral agent with an optionally substituted dibenzyl halogenophosphate derivative in the presence of a dehydrohalogenating agent.
[11] A pharmaceutical composition, which comprises the compound according to any one of those described in [1] to [9], or salt thereof.
[12] A pharmaceutical composition according to that described in [11], which is a growth inhibitor of cancer cells or virus-infected cells.
[13] A pharmaceutical composition according to that described in [11], which is a preventive or therapeutic agent for cancers or viral infections.
[14] A method for inhibiting the growth of cancer cells or virus-infected cells in a mammal, which comprises administering an effective amount of the compound according to any one of those described in [1] to [9], or a salt thereof to the mammal.
[15] A method for preventing or treating cancers or viral infections in a mammal, which comprises administering an effective amount of the compound according to any one of those described in [1] to [9], or a salt thereof to the mammal.

### Effects of the Invention

According to the present invention, the 5′-position dibenzyl monophosphate derivative of nucleoside-based anticancer agent or antiviral agent becomes more lipophilic than the corresponding nucleoside-based anticancer agent or antiviral agent, so that it can be orally administrated. After being absorbed in intestines, it passes through cell membrane of frequently dividing cancer cells or virus-infected cells without being affected by various hydrolytic metabolic enzymes in blood or liver (such as carboxyesterase, cytidine deaminase, nuclease, phosphatase, phosphodiesterase, and the like), and it is gradually hydrolyzed non-enzymatically in cell membrane or in cells. Subsequently, it undergoes enzymatic hydrolysis by phosphodiesterase to release the corresponding 5′-position monophosphate of nucleoside-based anticancer agent or antiviral agent. These 5′-position monophosphate derivatives are incorporated into DNA and RNA via nucleic acid biosynthetic route and exhibits a cytocidal effect by inhibiting the modification and extension of DNA and RNA, inhibiting the synthesis of corresponding protein, and inhibiting reverse transcriptase. Therefore, they can be expected to function as therapeutic or preventive agents for various cancers and viral infections. On the other hand, they can be expected to function as therapeutic agents that are also effective for cancer and virus-infected cells that have acquired resistance due to down-regulation of nucleoside kinase.

### Modes to Carry Out the Invention

Terms used in the specification and claims have following meanings, unless otherwise stated.

The compound of the present invention, or salt thereof

The compound of the present invention is represented by formula (I) as below,

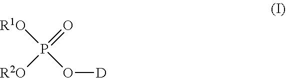

wherein, D is 5′-position moiety of nucleoside-based anticancer agent or antiviral agent, each of R¹ and R² is a benzyl group which may have a substituent or substituents, R¹ and R² may be the same or different.

The nucleoside-based anticancer agents shown as D include Cytarabine, Floxuridine, Pentostatin, Fludarabine, Cladribine, Gemcitabine, Clofarabine, Nelarabine, Trifluorothymidine (TFT), DFP-10917, Cordycepin, 8-Chloro-adenosine, RX-3117, Triciribine, Forodesine, 5-Fluoro-2′-deoxycytidine, Ribavirin, Acadecine, and the like. Their chemical structures are shown below as examples.

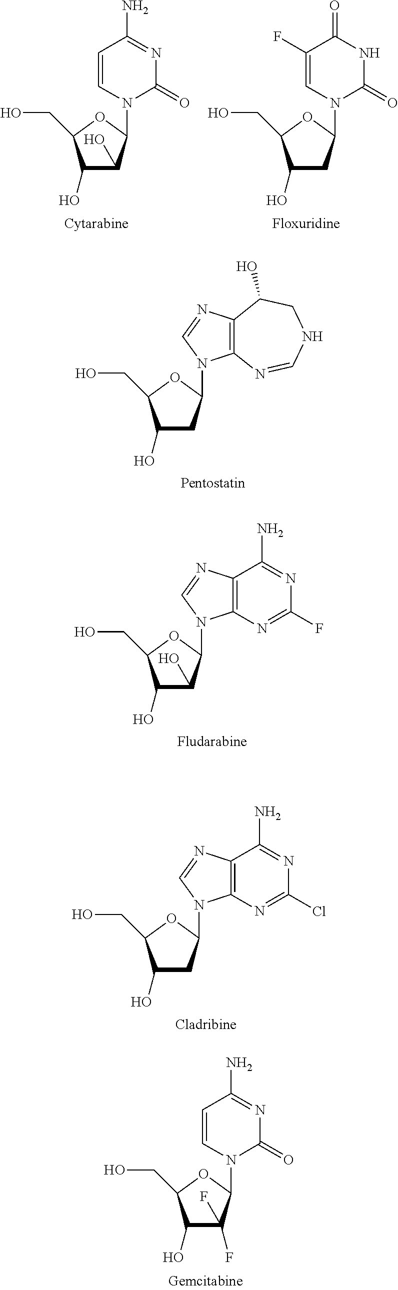

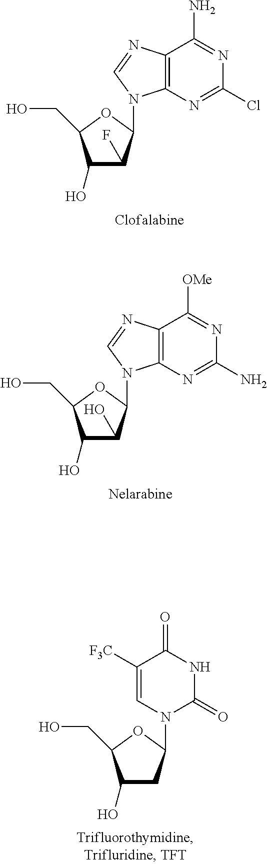

Besides, the nucleoside-based antiviral agents shown as D include Zidovudine, Lamivudine, Stavudine, Abacavir, Emtricitabine, Didanosine, Stavudine, and the like. Their chemical structures are shown below.

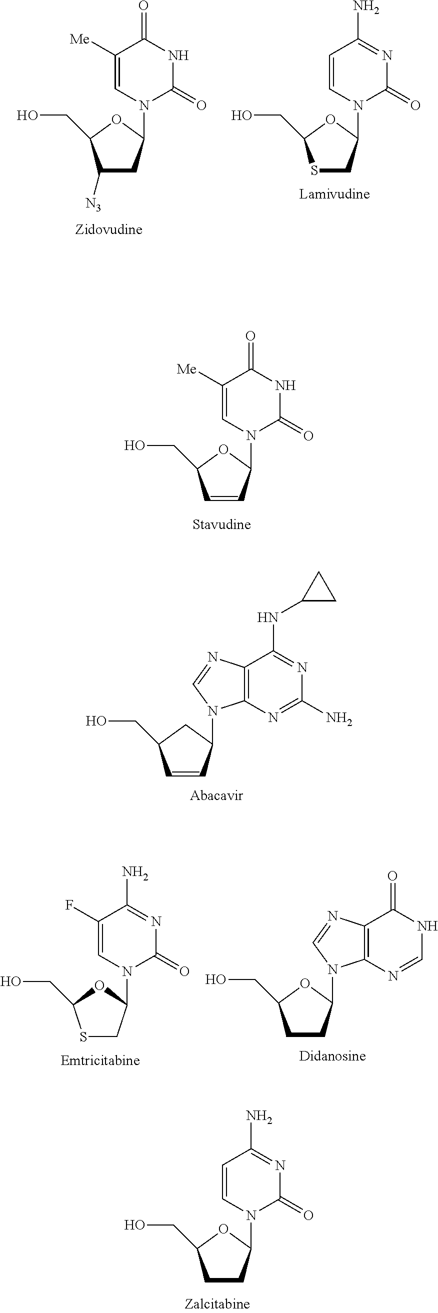

Examples of the compound represented by the formula (I) of the present invention include compounds represented by the following formulas (i) to (xvi), and the like.

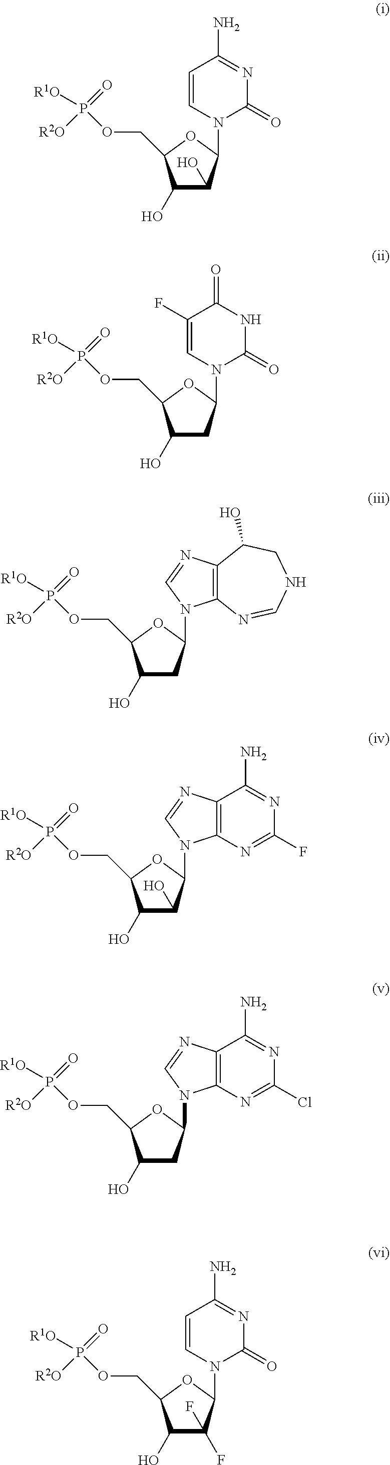

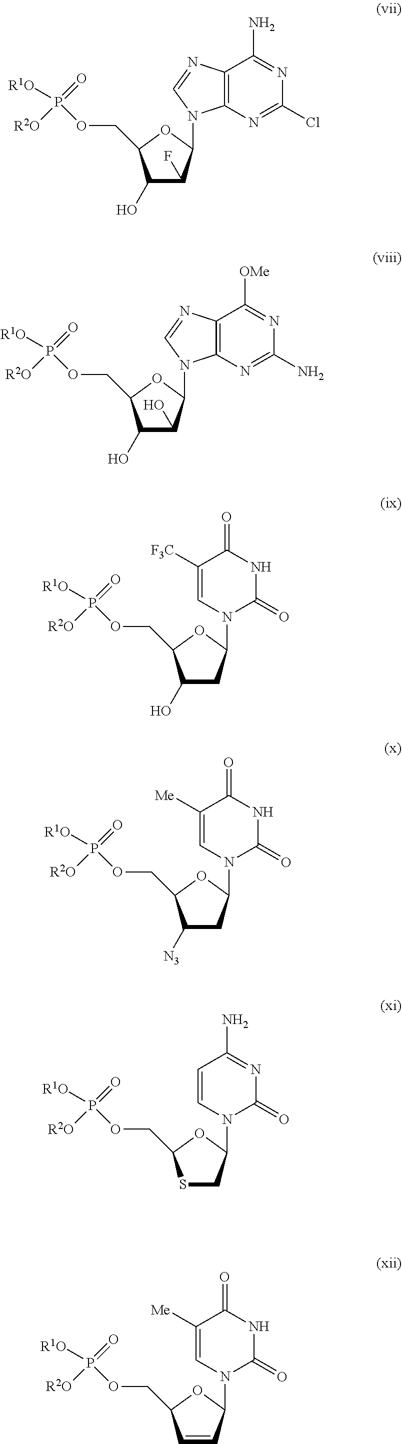

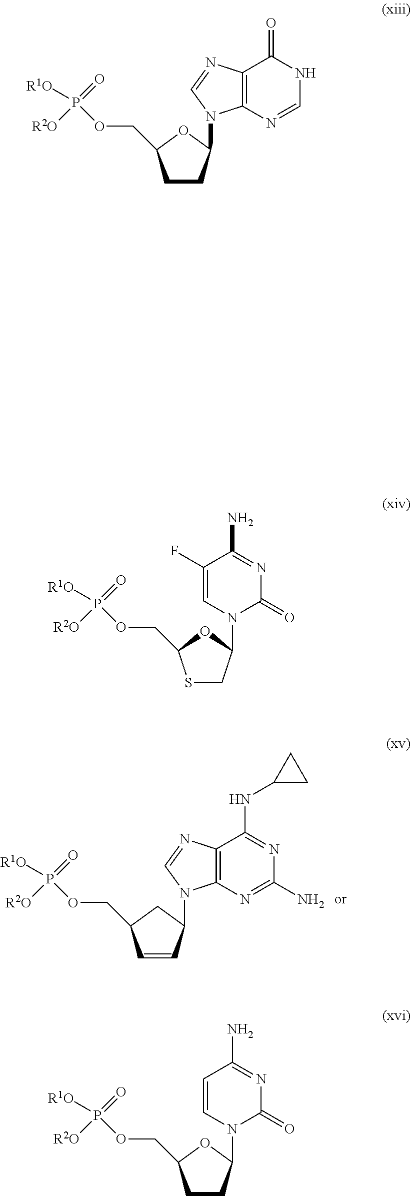

In the above formulas (i) to (xvi), each of R¹ and R² is a benzyl group which may have a substituent or substituents. R¹ and R² may be the same or different.

“A benzyl group which may have a substituent or substituents” refers to that it may or may not have a substituent or substituents. There may be 1 to 5 substituents, preferably 1 to 3 substituents at substitutable position of the benzyl group. When the number of substituents is 2 or more, the substituents may be the same or different. Examples of the substituents include alkyl group, halogen atom, cyano group, nitro group, and the like. Preferable examples of the substituents are alkyl group and halogen atom.

“Alkyl groups” refer to, unless otherwise limited, saturated aliphatic hydrocarbon groups, such as C₁ to C₂₀ straight or branched chains of alkyl groups or cyclic alkyl groups. Examples of the straight or branched chains of alkyl groups include C₁ to C₆ alkyl groups, such as methyl, ethyl, propyl, iso-propyl, butyl, sec-butyl, iso-butyl, tert-butyl, pentyl, hexyl groups, and the like, heptyl, 1-methylhexyl, 5-methylhexyl, 1,1-dimethylpentyl, 2,2-dimethylpentyl, 4,4-dimethylpentyl, 1-ethylpentyl, 2-ethylpentyl, 1,1,3-trimethylbutyl, 1,2,2-trimethylbutyl, 1,3,3-trimethylbutyl, 2,2,3-trimethylbutyl, 2,3,3-trimethylbutyl, 1-propylbutyl, 1,1,2,2-tetramethylpropyl, octyl, 1-methylheptyl, 3-methylheptyl, 6-methylheptyl, 2-ethylhexyl, 5,5-dimethylhexyl, 2,4,4-trimethylpentyl, 1-ethyl-1-methylpentyl, nonyl, 1-methyloctyl, 2-methyloctyl, 3-methyloctyl, 7-methyloctyl, 1-ethylheptyl, 1,1-dimethylheptyl, 6,6-dimethylheptyl, decyl, 1-methylnonyl, 2-methylnonyl, 6-methylnonyl, 1-ethyloctyl, 1-propylheptyl, n-nonyl, n-decyl groups, and the like, preferably, C₁ to C₆ alkyl groups. Preferable examples of C₁ to C₆ alkyl groups are methyl and ethyl groups. Examples of the cyclic alkyl groups include cyclopropyl, cyclobutyl, cyclopentyl, cyclohexyl, cycloheptyl, cyclooctyl groups, and the like. In addition, preferable examples of the cyclic alkyl groups are cyclopentyl and cyclohexyl groups.

“A halogen atom” refers to a fluorine atom, a chlorine atom, a bromine atom or an iodine atom, and the like. Preferable examples are a fluorine atom, a chlorine atom and a bromine atom.

Salts of the compound represented by formula (I) of the present invention may be any salts as long as they are pharmaceutically acceptable. Their examples include, but are not limited to, acid added salts including inorganic salts (such as hydrochloride, sulfate, hydrobromide, phosphate, etc.) and organic salts (such as acetate, trifluoroacetate, succinate, maleate, fumarate, propionate, citrate, tartrate, lactate, oxalate, methane sulfonate, p-toluene sulfonate, etc.), and the like.

The compound represented by formula (I) of the present invention may be crystal, which may be in single crystalline form or a mixture of multiple crystalline forms. The crystals can be produced by crystallization according to conventional methods.

In addition, the compound represented by formula (I) of the present invention may be a solvate (for example, a hydrate and the like). Both the solvate and non-solvate (for example, a non-hydrate and the like) are included in the compound represented by formula (I).

The 5′-position dibenzyl monophosphate derivative of nucleoside-based anticancer agent or antiviral agent of the present invention can be a prodrug of 5′-position monophosphate of nucleoside-based anticancer agent or antiviral agent.

The 5′-position dibenzyl monophosphate derivative of nucleoside-based anticancer agent or antiviral agent is remarkably stable with respect to hydrolytic metabolic enzymes, such as carboxyl esterase, cytidine deaminase, nuclease, phosphatase, phosphodiesterase, and the like. The 5′-position dibenzyl monophosphate derivative of nucleoside-based anticancer agent or antiviral agent absorbed from intestines is hydrolyzed non-enzymatically or enzymatically in cancer cells or virus-infected cells to release corresponding 5′-position monophosphate of nucleoside-based anticancer agent or antiviral agent. These 5′-position monophosphate derivatives are incorporated into DNA and RNA via nucleic acid biosynthetic route, and are expected to exhibit a cytocidal effect by inhibiting the modification and extension of DNA and RNA, inhibiting the synthesis of corresponding protein, or inhibiting reverse transcriptase.

Therefore, the 5′-position dibenzyl monophosphate derivative of nucleoside-based anticancer agent or antiviral agent according to the present invention is expected to have remarkable stability with respect to hydrolytic metabolic enzyme and to become a prodrugs of various 5′-position monophosphates of nucleoside-based anticancer agents or antiviral agents.

### Methods for Producing the Compound Represented by Formula (i) of the Present Invention

The compound represented by formula (I) of the present invention can be produced according to, for example, the following methods or other similar ones.

### Method A

The compound of formula (I) or a salt thereof can be produced according to conventional methods or their similar ones (see Bulletin of the Chemical Society, 1969, 42(12), 3505-8, Nucleic Acids Research, 1984, 12, 5025-36, Chemical & Pharmaceutical Bulletin, 1995, 43(2), 210-215, WO-201111317). For example, a commercially available nucleoside-based anticancer agent or antiviral agent (sometimes called as nucleosides) is activated with phosphorus oxychloride in an appropriate solvent, and then reacted with optionally substituted benzyl alcohol in the presence of a dehydrohalogenating agent. The targeted 5′-position dibenzyl monophosphate derivative of nucleoside-based anticancer agent or antiviral agent (see formula (I)) can be obtained.

### Method B

Regarding the compound of formula (I) or a salt thereof, for example, a commercially available nucleoside-based anticancer agent or antiviral agent is reacted with dibenzyl chlorophosphate derivative in an appropriate solvent in the presence of a dehydrohalogenating agent. The targeted 5′-position dibenzyl phosphate derivative of nucleoside-based anticancer agent or antiviral agent (see formula (I)) can be obtained.

### (Dehydrohalogenating Agents)

Examples of dehydrohalogenating agents used include organic bases and inorganic bases. Examples of the organic bases include, but are not limited to, triethylamine, N, N-diisopropylethylamine, pyridine, 4-dimethylaminopyridine (DMAP), n-butyllithium and potassium tert-butoxide. Examples of the inorganic bases include, but are not limited to, sodium hydride, sodium carbonate, sodium bicarbonate, potassium carbonate, potassium bicarbonate and cesium carbonate. The amount of the base used is preferably 2 mol or more of the compound of starting material. Furthermore, a range of usually 2.0 to 50.0 mol, preferably the range of 5.0 to 20.0 mol, and more preferably the range of 5.0 to 10.0 mol can be exemplified with respect to 1 mol of the compound of starting material.

### (Reaction Solvents)

From the viewpoints of smooth progress of reactions and the like, it is preferred that the reactions of the present invention are carried out in a solvent. Any solvent can be used for the reactions of the present invention as long as the reactions proceed.

Examples of the reaction solvents include phosphates such as trimethyl phosphate, triethyl phosphate, tributyl phosphate, triphenyl phosphate, tricresyl phosphate, and the like in the case of Method A, and pyridine and the like in the case of Method B. The amount of the solvents used may be any amount as long as the reactions proceed. The amount of the solvents used in the reactions of the present invention can be adjusted appropriately by a person skilled in the art.

### (Reaction Temperature)

Reaction temperature of the present invention is not particularly limited. In one embodiment, from the viewpoints of yield improvement, by-product control, economic efficiency, and the like, the range of −20 to 50° C. (minus 20 to plus 50° C.), preferable range of −10 to 30° C. (minus 10 to plus 30° C.), more preferable range of −10 to 20° C. (minus 10 to plus 20° C.), even more preferable range of −5 to 15° C. (minus 5 to plus 15° C.) and especially preferable range of −5 to 10° C. (minus 5 to plus 10° C.) can be mentioned as examples.

### (Reaction Time)

Reaction time of the present invention is not particularly limited. In one embodiment, from the viewpoints of yield improvement, by-product control, economic efficiency, and the like, the range of 0.5 to 120 hours, preferable range of 1 to 72 hours, more preferable range of 1 to 48 hours, even more preferable range of 1 to 24 hours can be mentioned as examples. However, reaction time of the present invention can be adjusted appropriately by a person skilled in the art.

### Pharmaceutical Compositions of the Present Invention

The compound represented by formula (I) of the present invention can be used as a safe medicine for mammals (such as humans, monkeys, cats, pigs, horses, cattle, mice, rats, guinea pigs, dogs, rabbits, and the like) as it is or as a pharmaceutical composition mixed with pharmaceutically acceptable carriers according to conventional methods.

Regarding the said pharmaceutically acceptable carriers, various conventional organic or inorganic substances can be used as formulation materials. Examples include solid formulations, such as excipients, lubricants, binding agents and disintegrating agents; liquid formulations, such as solvents, solubilizing agents, suspending agents, tonicity agents and buffers, and the like. Furthermore, formulation additives, such as preservative agents, antioxidant agents, coloring agents, sweetening agents, and the like can also be used when necessary.

Regarding dosage forms of the pharmaceutical compositions, oral preparations such as tablets, capsules (including soft capsules and microcapsules), granules, powders, syrups, emulsions, suspensions, sustained-release preparations, and the like, can be mentioned as examples. These can be administered orally and safely. However, they are not limited to these examples, because liquid formulations are also possible.

The pharmaceutical compositions can be produced according to conventional methods in technical field of formulation, for example, the methods described in The Japanese Pharmacopeia, and the like.

### Use of the Compound Represented in Formula (i) of the Present Invention

The compound represented in formula (I) of the present invention can be used in many therapeutic and preventive ways. In a preferable embodiment, it is used for treatment of indications corresponding to each nucleoside-based anticancer agent or antiviral agent. For example, in case of 5′-position dibenzyl monophosphate derivative of Gemcitabine (see structure vi in the above figure), preferable indications are non-small cell lung cancer, pancreatic cancer, biliary tract cancer, urothelial cancer, inoperable or recurrent breast cancer, ovarian cancer that has worsened after cancer chemotherapy, relapsed or refractory malignant lymphoma, and the like.

Suitable pharmaceutical compositions used in the present invention comprise active ingredients in such effective amounts so that purposes of treating and/or preventing the symptoms (for example, hematological abnormality (such as sickle cell anemia), MDS and/or cancer (for example, NSCL)) can be achieved.

The pharmaceutical compositions used in the present invention are provided as a dosage forms for oral administrations. The pharmaceutical compositions provided in this specification can be provided in solid, semi-solid, or liquid form for oral administrations. As used in this specification, oral administration also includes buccal, lingual and sublingual administrations. Suitable oral dosage forms include, but are not limited to, tablets, capsules, pills, troches, medicinal candy, aroma preparations, cachets, pellets, drug-added chewing gum, granules, bulk powders, foamed formulations, or non-foamed powders or granules agents, solutions, emulsions, suspensions, solutions, wafers, sprinkles, elixirs and syrups. In addition to the active ingredient(s), pharmaceutical compositions comprise, but not limited to, binders, fillers, diluents, disintegrants, wetting agents, lubricants, glidants, colorants, pigment migration inhibitors, sweeteners and flavoring agents. Moreover, they may also comprise one or more pharmaceutically acceptable carriers or excipients.

Amounts of the compound represented by formula (I) of the present invention in pharmaceutical compositions or dosage forms may be, for example, in any one of the ranges of about 1 to 2,000 mg, about 10 to 2,000 mg, about 20 to 2,000 mg, about 50 to 1,000 mg, about 100 to 500 mg, about 150 to 500 mg, or about 150 to 250 mg.

When using the compound of the present invention as an anticancer agent, its effective dosages can be properly chosen according to character and stage of cancer, therapeutic strategy, extent of metastasis, amount of tumor, body weight, age, sex and background of genetic race of patients, and the like. Pharmaceutically effective dosages are generally determined according to factors such as clinical observation of symptoms, stage of cancer, and the like. Daily dosage, in case of administration to human as an example, is in the range of about 0.01 to 10 mg/kg (about 0.5 to 500 mg for a 60 kg adult), preferably about 0.05 to 5 mg/kg and more preferably about 0.1 to 2 mg/kg. The administration may be performed once or divided into multiple times.

### EXAMPLES

The examples provided below further illustrate the present invention. However, the present invention is not limited in any way by them.

In following examples, room temperature refers to about 15 to 30° C. The determinations of ¹H-NMR and ¹³C-NMR were conducted with a JNM-ECZ 400R instrument (JEOL), in which DMSO-d₆ or CD₃OD was used as a solvent, and chemical shifts (δ) from tetramethylsilane, an internal standard, are shown in ppm. Other terms used in the specification have the following meanings. s: singlet; d: doublet; t: triplet; m: multiplet; br: broad; br s: broad singlet; J: constant of J-coupling. In addition, mass determination of each compound was conducted with a Yamazen Smart Flash MS system.

### Example 1

Activation of Nucleosides by Phosphorus Oxychloride and Subsequent Condensation with Benzyl Alcohols

A nucleoside (0.5 mM) was suspended in about 1 mL of triethyl phosphate at room temperature, added with 93 μL of phosphorus oxychloride (about 2 times mol to the starting material) while cooling at 0° C. and stirred for about 2 hours. Then, the solution was added with a corresponding benzyl alcohol (about 10 times mol) and about 0.4 mL of pyridine (about 10 times mol) and stirred further for 1 hour while cooling at 0° C. The reaction solution was poured into a mixture of ethyl acetate-water, neutralized with dilute solution of sodium hydrogen carbonate and extracted with ethyl acetate. The extract was washed with saturated brine and dried over anhydrous magnesium sulfate. After insoluble materials were removed, the extract was concentrated to dryness under reduced pressure. An oily residue obtained was separated and purified with a silica gel column (Yamazen Smart Flash MS system). The targeted 5′-position dibenzyl monophosphate derivative of the nucleoside was obtained. This is referred to as synthetic method A hereafter.

### Example 2

Condensation of Nucleosides with Dibenzyl Chlorophosphate Derivatives

A nucleoside (0.5 mM) was suspended in about 1.0 mL of anhydrous pyridine at room temperature, added with about 0.25 mL of a corresponding dibenzyl chlorophosphate derivative (about 1.2 times mol) while cooling at 0° C. and stirred for about 1 hour. Then, the reaction solution was poured into a mixture of ethyl acetate-water, neutralized with dilute solution of sodium hydrogen carbonate, and extracted with ethyl acetate. The extract was washed with saturated brine and dried over anhydrous magnesium sulfate. After insoluble materials were removed, the extract was concentrated to dryness under reduced pressure. An oily residue obtained was separated and purified with a silica gel column (Yamazen Smart Flash MS system). The targeted 5′-position dibenzyl monophosphate derivative of the nucleoside was obtained. This is referred to as synthetic method B hereafter.

The separation systems of silica gel column, separation yields, data obtained from instrumental analysis and distribution coefficients of the compounds (1) to (4) which are compounds of 5′-position dibenzyl monophosphate of nucleosides synthesized according to the above synthetic methods A or B are shown as below.

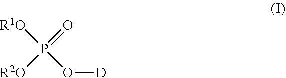

Compound (1): O,′-Di (4-fluoro)benzyl 2′-deoxy-2′,2′-difluoro-5′-cytidylate (D=2′-Deoxy −2′,2′-difluorocytidin −5′-yl, R¹=R²=4-Fluorobenzyl in formula (I)): (Synthetic methods A and B), eluent system in silica gel column: ethyl acetate/methanol
Yield:15% (synthetic method A), 50% (synthetic method B)
Mass=m/e 560.2 (M⁺+1) (calcd. for C₂₃H₂₂F₄N₃O₇P, MW=559.11)

¹H-NMR (CD₃OD) δ: 4.02-4.07 (1H, m), 4.10-4.23 (1H, m), 4.25-4.42 (2H, m), 5.06 (2H, s), 5.08 (2H, s), 5.83 (1H, d, J=7.7 Hz), 6.23 (1H, br t, J=8.3 Hz), 7.05-7.13 (4H, m), 7.35-7.42 (4H, m), and 7.52 (1H, d, J=7.8 Hz) ppm.

¹ H-NMR (DMSO-d₆) δ: 3.95-4.05 (1H, m), 4.10-4.25 (1H, m), 4.25-4.32 (2H, m), 5.03 (2H, br s), 5.05 (2H, br s), 5.73 (1H, d, J=7.3 Hz), 6.17 (1H, br t), 6.45 (1H, d, J=6.4 Hz), 7.25-7.35 (4H, m), 7.37-7.42 (4H, m), 7.4 (2H, br), and 7.49 (1H, d, J=7.7 Hz) ppm.

¹³C-NMR (CD₃OD) δ: 67.0 (4.8 Hz), 70.3 (t, 4.8 Hz), 71.0, 71.2, 71.5, 80.1, 96.6, 116.3, 116.5, 120.8, 123.4, 126.0, 131.4, 131.5, 132.9(6.8 Hz), 133.0 (6.8 Hz), 142.4, 157.4, 163.1, 165.5, and 167.4 ppm.
Distribution coefficient: log P(n-octanol/_{PBS})=2.288
   Compound (2): O,O′— Di (4-chloro)benzyl 2′-deoxy-2′,2′-difluoro-5′-cytidylate (D=2′-Deoxy-2′,2′-difluorocytidin -5′-yl, R¹=R²=4-Chlorobenzyl in formula (I)): (Synthetic method A), eluent system in silica gel column: ethyl acetate/methanol
Yield: 15.4%
Mass=m/e 592.2 (M⁺+1) (calcd. for C₂₃H₂₂C₁₂F₂N₃O₇P, MW=591.05

¹ H-NMR (CD₃OD) δ: 4.03-4.10 (1H, m), 4.14-4.25 (1H, m), 4.30-4.45 (2, m), 5.06 (2H, s), 5.09 (2H, s), 5.83 (1H, d, J=7.3 Hz), 6.23 (1H, br t, J=8.2 Hz), 7.25-7.40 (8H, m), and 7.50 (1H, d, J=7.8 Hz) ppm.

¹-H-NMR (DMSO-d₆) δ: 3.95-4.05 (1H, m), 4.10-4.25 (1H, m), 4.25-4.32 (2H, m), 5.04 (2H, br s), 5.06 (2H, br s), 5.74 (1H, d, J=7.8 Hz), 6.20 (1H, br t), 6.45 (1H, d, J=6.4 Hz), 7.34-7.40 (4H, m), 7.40-7.46 (6H, m), and 7.49 (1H, d, J=7.8 Hz) ppm.

¹³C-NMR (CD₃OD) δ: 67.2 (5.8 Hz), 70.3 (t, 4.8 Hz), 71.1, 71.4, 71.6, 80.2, 96.7, 120.9, 123.5, 126.1, 129.9, 130.8, 135.8 (6.8 Hz), 142.5, 157.5, and 167.5 ppm.
Distribution coefficient: log P(n-octanol/_{PBS})=3.062
   Compound (3): O,O′— Di(4-bromo)benzyl 2′-deoxy-2′,2′-difluoro-5′-cytidylate (D=2′-Deoxy-2′,2′-difluorocytidin -5′-yl, R¹=R²=4-Bromo-benzyl in formula (I)): (Synthetic method A), eluent system in silica gel column: ethyl acetate/methanol,
Yield: 15.1% (1.0 mM scale)
Mass=m/e 682.1 (M⁺+1) (calcd. for C₂₃H₂₂Br₂F₂N₃O₇P, MW=681.22

¹H-NMR (CD₃OD) δ: 4.05-4.10 (1H, m), 4.15-4.25 (1H, m), 4.30-4.45 (2, m), 5.04 (2H, s), 5.06 (2H, s), 5.84 (1H, d, J=7.3 Hz), 6.24 (1H, br t, J=8 Hz), 7.23-7.30 (4H, m), and 7.45-7.53 (5H, m) ppm.

¹³C-NMR (CD₃OD) δ: 67.2 (4.8 Hz), 70.2 (t, 4.9 Hz), 71.0, 71.3, 71.5, 80.1, 96.7, 120.8, 123.4, 123.7, 126.0, 130.9, 132.8, 136.1 (6.8 Hz), 142.4, 157.6, and 167.5 ppm.
Distribution coefficient: log P(n-octanol/_{PBS})=3.325
   Compound (4): O,O′— Di(4-fluoro)benzyl 5-fluoro-2′-deoxy-5′-cytidylate (D=5-Fluoro-2′-deoxycytidin-5′-yl, R¹ =R²=4-Fluorobenzyl in formula (I)): (Synthetic method A), eluent system in silica gel column: ethyl acetate/methanol,
Yield: 13.0%
Mass=m/e 542.2 (M⁺+1) (calcd. for C₂₃H23F3N₃O₇P, MW=541.12)

¹H-NMR (CD₃OD) δ: 2.00-2.10 and 2.30-2.40 (each 1H, each m), 4.00-4.10 (1H, m), 4.18-4.32 (3H, m), 5.05 (2H, br s), 5.11 (2H, br s), 6.17 (br t, J=6.0 Hz), 7.00-7.15 (4H, m), 7.30-7.45 (4H, m), and 7.87 (1H, d, J=6.4 Hz) ppm.
Distribution coefficient: log P(n-octanol/_{PBS})=2.196

### Test Example 1

Stability of 5′-Position Dibenzyl Monophosphate Derivative of Nucleoside-Based Anticancer Agent or Antiviral Agent with Respect to Cytidine Deaminase

About 1 mg of a 5′-position dibenzyl monophosphate derivative of nucleoside-based anticancer agent or antiviral agent (see formula (I)) obtained was dissolved in 1 mL of acetonitrile. 10 μL of the solution was added with 1 mL of PBS. 10 μL of PBS solution of cytidine deaminase was added to the solution and stirred at 37° C. for about 30 minutes to 1 hour. 1 mL of acetonitrile was added to the reaction solution and separated by centrifugation. The supernatant was analyzed with HPLC. As examples, the analytical results of Cytidine, Gemcitabine and O,O′— Di(4-fluoro)benzyl 2′-deoxy-2′, 2′-difluoro −5′-cytidylate (compound (1)) are shown in Table 1.

Cytidine deaminase: CDA (1-146aa), Human, His-tagged, Recombinant cytidine deaminase (ATGen)

HPLC conditions:
Column: ZORBAX Bonus-RP
   4.6 mm×250 mm, particle size: 5μm
Elution: eluate A=Purified water containing 10 mM ammonium formate
   eluate B=Acetonitrile
   Gradient mode: A: B=99:1→20:80/30 minutes
Flow rate: 1.0 mL/min
Oven temperature: 40° C.
Detection: UV240 nm

TABLE 1

| Starting material | Change in HPLC pattern |
|---|---|
| Cytidine | The peak of the starting material disappeared |
| | completely after 30 minutes. |
| Gemcitabine | The peak of the starting material disappeared |
| | completely after 30 minutes. |
| Compound (1) | Almost no change in the peak of the starting |
| | material was confirmed even after 1 hour. |

Accordingly, it has been confirmed that the 5′-position dibenzyl monophosphate derivative of nucleoside-based anticancer agent or antiviral agent according to the present invention was extremely stable with respect to cytidine deaminase under physiological conditions. On the other hand, Cytidine and Gemcitabine were not stable under the above reaction conditions and disappeared completely.

### Test Example 2

### Non-Enzymatic and Enzymatic Hydrolysis of 5′-Position Dibenzyl Monophosphate Derivative of Nucleoside-based Anticancer Agent or Antiviral Agent

About 1 mg of a 5′-position dibenzyl monophosphate derivative of nucleoside-based anticancer agent or antiviral agent (see formula (I), for example, O,O′— Di (4-fluoro)benzyl 2′-deoxy-2′, 2′-difluoro-5′-cytidylate, compound (1)) obtained was dissolved in 1 mL of acetonitrile. 10 μL of the solution was added to 1 mL of PBS solution. 10 μL each of the following PBS solutions of hydrolytic enzymes was added and stirred at 37° C. for about 1 hour. 1 mL of acetonitrile was added to the reaction mixtures and centrifuged. The results of HPLC analysis of each supernatant are shown in Table 2. HPLC conditions for the determination were the same as those in test example 1.

TABLE 2

| Hydrolytic enzymes | Change in HPLC pattern |
|---|---|
| Phosphodiesterase I | Almost no change in the peak of the starting |
| | material was confirmed even after 1 hour. |
| Phosphodiesterase II | Almost no change in the peak of the starting |
| | material was confirmed even after 1 hour. |
| Nuclease | Almost no change in the peak of the starting |
| | material was confirmed even after 1 hour. |
| Phospholipase CB1 | Almost no change in the peak of the starting |
| | material was confirmed even after 1 hour. |
| Phospholipase CD1 | Almost no change in the peak of the starting |
| | material was confirmed even after 1 hour. |
| Phospholipase CG1 | Almost no change in the peak of the starting |
| | material was confirmed even after 1 hour. |
| Alkaline Phosphatase I | Almost no change in the peak of the starting |
| | material was confirmed even after 1 hour. |
| Alkaline Phosphatase L | Almost no change in the peak of the starting |
| | material was confirmed even after 1 hour. |
| Acid Phosphatase | Almost no change in the peak of the starting |
| | material was confirmed even after 1 hour. |

The enzymes used in the test were Phosphodiesterase I (Crotalus adamanteus Venom: WOR), Phosphodiesterase II (Bovine spleen: WOR), Nuclease (Staphylococcus: SIGMA), Phospholipase CB1 (Human recombinant: ABV), Phospholipase CD1 (Human recombinant: ABV), Phospholipase CG1 (Human recombinant: ABV), Alkaline Phosphatase I (OPCA00948: Recombinant human Intestinal-type: AVIVA Systems Biolog), Alkaline Phosphatase L (OPCA00950: Recombinant human, tissue-nonspecific isozyme: AVIVA Systems Biolog) and Acid Phosphatase (1-158aa: Human, His-tagged, Recombinant, E. Coli: ATGen).

Therefore, 5′-position dibenzyl monophosphate derivative of nucleoside-based anticancer agent or antiviral agent (see formula (I)) was remarkably stable in presence of any hydrolytic enzyme. Meanwhile, these 5′-position dibenzyl monophosphate derivatives of nucleoside-based anticancer agent or antiviral agent (for example, O,O′— Di (4-fluoro)benzyl 2′-deoxy-2′,2′-difluoro-5′-cytidylate, compound (1)) is gradually hydrolyzed under physiological conditions (for example, in PBS solution at 37° C.) to give the corresponding 5′-position monobenzyl monophosphate derivative. However, the monobenzyl derivative is smoothly hydrolyzed under physiological conditions by Phosphodiesterase I to produce the corresponding 5′-position monophosphate (for example, 2′-Deoxy-2′,2′-difluoro-5′-cytidylic acid: Gemcitabine 5′-monophosphate) almost quantitatively.

### Test Example 3

### Biological Activity of 5′-Position Dibenzyl Monophosphate Derivative of Nucleoside-Based Anticancer Agent or Antiviral Agent

Each of the test compounds (DMSO solutions in various concentrations) was added to 100 μL of a culture solution containing human pancreatic cancer cells (MIA-Paca-2) (cell count: about 5×10³ cells) and cultured for 3 days. The cell growth inhibitory effect was investigated by fluorescence color development using alamarBlue reagent. Each of their IC₅₀ values was determined. The results are shown in Table 3.

TABLE 3

| Compound | IC₅₀ (μM) |
|---|---|
| O,O′-Di (4-fluoro) benzyl 2′-deoxy-2′,2′-difluoro-5′-cytidylate | 9.8 |
| (Compound (1)) | |
| O,O′-Di (4-chloro) benzyl 2′-deoxy-2′,2′-difluoro-5′-cytidylate | 18.0 |
| (Compound (2)) | |
| O,O′-Di (4-bromo) benzyl 2′-deoxy-2′,2′-difluoro-5′-cytidylate | 21.8 |
| (Compound (3)) | |
| 2′-Deoxy-2′,2′-difluorocytidine (Gemcitabine) | 7.4 |

As shown above, the 5′-position dibenzyl monophosphate derivative of nucleoside-based anticancer agent or antiviral agent (see formula (I)) showed the similar biological activities with the nucleoside-based anticancer agent s or antiviral agents which were used as starting materials.

### INDUSTRIAL APPLICABILITY

According to the present invention, medicines that may replace nucleoside-based anticancer agent or antiviral agents clinically used as therapeutic or preventive agents for various cancers and viral infections can be provided for clinical practice.

1. A compound represented by formula (I), or salt thereof, 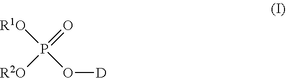
wherein D is 5′-position moiety of nucleoside-based anticancer agent or antiviral agent, and R¹ and R² are each a benzyl group that may have the same substituent or different substituents.

2. The compound according to claim 1, or salt thereof, wherein each of the R¹ and R² is a benzyl group which may have an alkyl or a halogen atom as a substituent.

3. The compound according to claim 2, or salt thereof, wherein the alkyl is C₁ to C₆ alkyl group.

4. The compound according to claim 2, or salt thereof, wherein the alkyl is methyl group or ethyl group.

5. The compound according to claim 2, or salt thereof, wherein the halogen atom is a fluorine atom, a chlorine atom or a bromine atom.

6. The compound according to claim 1, or salt thereof, wherein the R¹ and R² are benzyl groups.

7. The compound according to claim 1, or salt thereof, wherein the nucleoside-based anticancer agent shown as D is Cytarabine, Floxuridine, Pentostatin, Fludarabine, Cladribine, Gemcitabine, Clofarabine, Nelarabine, Trifluorothymidine, DFP-10917, Cordycepin, 8-Chloroadenosine, RX-3117, Triciribine, Forodesin, 5-Fluorodeoxycytidine, Ribavirin or Acadesine.

8. The compound according to claim 1, or salt thereof, wherein the antiviral agents shown as D is Zidovudine, Lamivudine, Stavudine, Abacavir, Emtricitabine, Didanosine or Stavudine.

9. The compound according to claim 1, or salt thereof, wherein the compound is

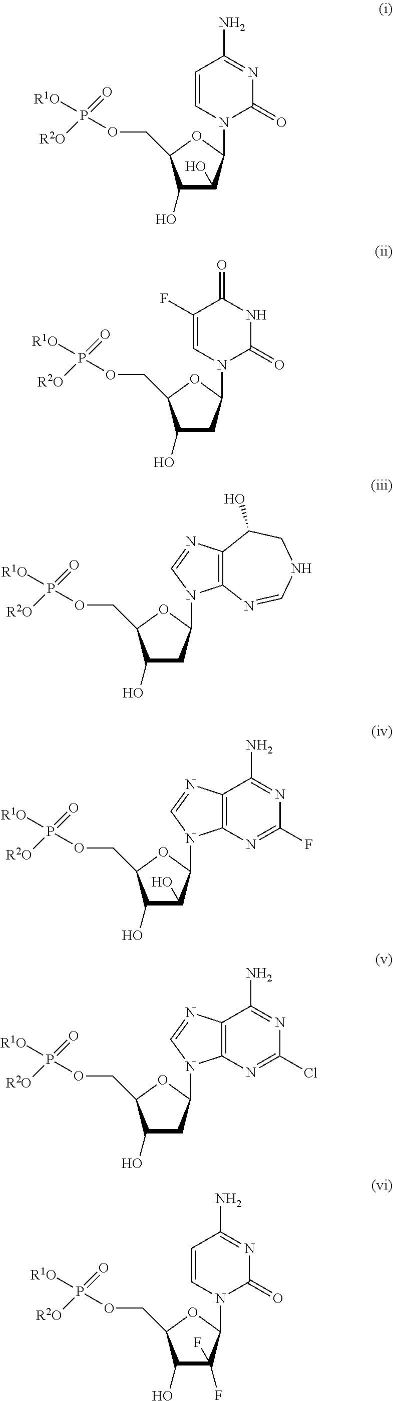

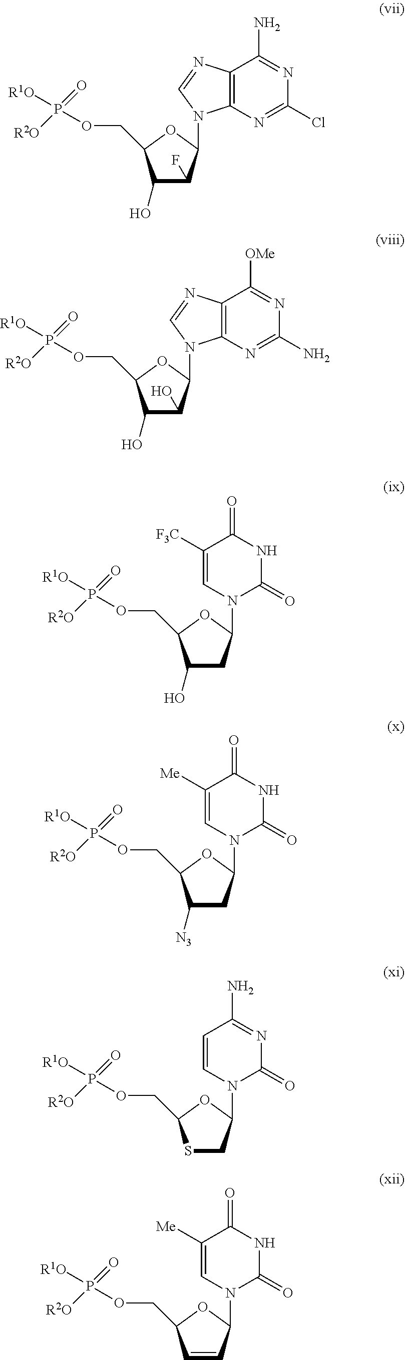

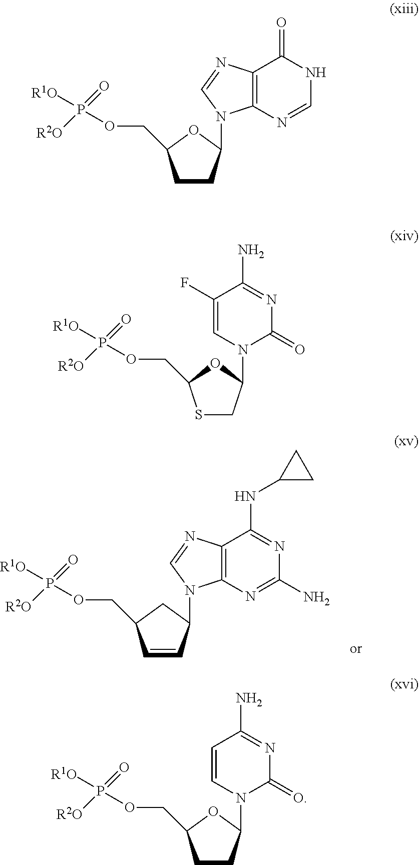

10. A method for producing the compound according to claim 1, or salt thereof, which comprises reacting a nucleoside-based anticancer agent or antiviral agent with phosphorus oxychloride and then reacting with an optionally substituted benzyl alcohol in the presence of a dehydrohalogenating agent, or comprises reacting a nucleoside-based anticancer agent or antiviral agent with an optionally substituted dibenzyl halogenophosphate derivative in the presence of a dehydrohalogenating agent.

11. A pharmaceutical composition, which comprises the compound according to claim 1, or salt thereof.

12. A pharmaceutical composition according to claim 11, which is a growth inhibitor of cancer cells or virus-infected cells.

13. A pharmaceutical composition according to claim 11, which is a preventive or therapeutic agent for cancers or viral infections.

14. A method for inhibiting the growth of cancer cells or virus-infected cells in a mammal, which comprises administering an effective amount of the compound according to claim 1, or a salt thereof to the mammal.

15. A method for preventing or treating cancers or viral infections in a mammal, which comprises administering an effective amount of the compound according to claim 1, or a salt thereof to the mammal.
